(19) United States
(12) Patent Application Publication
(10) Pub. No.: US 2024/0269003 A1
(43) Pub. Date: Aug. 15, 2024

(54) METHOD FOR PROVIDING CONTROL DATA FOR AN OPHTHALMOLOGICAL LASER OF A TREATMENT APPARATUS FOR AVOIDING OPTICAL ABERRATIONS

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/441,479

(22) Filed: Feb. 14, 2024

(30) Foreign Application Priority Data
Feb. 15, 2023 (DE) 10 2023 103 700.5

(51) Int. Cl. A61F 9/008

(52) U.S. Cl. CPC A61F 9/00804; A61F 9/00827; A61F 2009/00848; A61F 2009/00872

(57) ABSTRACT

The invention relates to a method for providing control data for an ophthalmological laser (12) of a treatment apparatus (10) for avoiding optical aberrations. As the steps, the method includes ascertaining (S10) first aberration values from a predetermined wavefront measurement of an eye, which has a first extension (32), wherein a first refractive power error is determined from the first aberration values; ascertaining (S12) second aberration values from a subset of the predetermined wavefront measurement, which has a second extension (34), wherein the second extension (34) is smaller than the first extension (32), wherein a second refractive power error is determined from the second aberration values; ascertaining (S14) a difference between the first and the second refractive power error; ascertaining (S16) an aberration-corrected refractive power change by subtracting the ascertained difference of refractive power errors from a predetermined subjective refractive power correction, which is predetermined from a glasses correction measurement; and providing (S18) the control data for the ophthalmological laser (12), which includes the aberration-corrected refractive power change.

### FIELD

The invention relates to a method for providing control data for an ophthalmological laser of a treatment apparatus for avoiding optical aberrations. In addition, the invention relates to a treatment apparatus with at least one eye surgical laser and at least one control device for performing the method, to a computer program and to a computer-readable medium.

### BACKGROUND

Treatment apparatuses and methods for controlling ophthalmological lasers for correcting an optical visual disorder and/or pathologically and/or unnaturally altered areas of the cornea are known in the prior art. Therein, pulsed lasers and a beam focusing device can for example be formed such that laser pulses effect a photodisruption and/or photoablation in a focus situated within the organic tissue, to remove a tissue, in particular a tissue lenticule, from the cornea.

If a treatment of an eye, in particular of the cornea, is determined exclusively based on a subjective refractive power correction or glasses correction, which can be measured by means of a phoropter, optical aberrations, in particular higher order aberrations, are not taken into account, since they cannot be captured by the phoropter measurement. In order to determine optical aberrations, in particular higher order aberrations, wavefront measurements are therefore usually performed, in particular by means of an aberrometer. However, combining the refractive power change, which has been determined from the subjective refractive power correction, with the aberration values, which have been determined from the wavefront measurement, is aggravated since reference centers of the glasses correction measurement and the wavefront measurement can deviate from each other. For example, the reference center of the glasses correction measurement is the visual axis and the reference center of the wavefront measurement is the pupil or the pupil center, wherein they can be shifted to each other in the eye.

Therefore, the object of the invention is in providing control data, with which optical aberrations in the correction of a cornea can be reduced or avoided.

### SUMMARY

This object is solved by the independent claims. Advantageous embodiments of the invention are disclosed in the dependent claims, the following description as well as the figures.

The invention is based on the idea that the same wavefront measurement is analyzed for two different extensions or diameters. One time for the full wavefront and the other time for a subset thereof. Therein, a refraction difference, which one obtains for the respective extension, corresponds to an effect, which is generated by higher order aberrations. Thus, the expected optical effect of higher order aberrations in the refraction can be calculated and be modified from the predetermined manifest refraction (glasses correction). In this manner, parabolic low order terms of the modified wavefront aberration can be determined for the entire wavefront range.

An aspect of the invention relates to a method for providing control data for an ophthalmological laser of a treatment apparatus for avoiding optical aberrations, wherein the method can be performed by a control device. By a control device, an appliance or an appliance component, in particular a processor, preferably microprocessor, is provided, which can perform the following steps.

Ascertaining first aberration values from a predetermined wavefront measurement of an eye, wherein the wavefront measurement has a first extension, wherein a first refractive power error is determined from the first aberration values, ascertaining second aberration values from a subset of the predetermined wavefront measurement, which has a second extension, wherein the second extension is smaller than the first extension, wherein a second refractive power error is determined from the second aberration values, and ascertaining a difference between the first and the second refractive power error are effected.

Furthermore, ascertaining an aberration-corrected refractive power change by subtracting the ascertained difference of refractive power errors from a predetermined subjective refractive power correction, which is predetermined from a glasses correction measurement, and providing the control data for the ophthalmological laser, which includes the aberration-corrected refractive power change, are effected.

In other words, a wavefront measurement of the eye and a subjective refractive power correction, which is performed from a glasses correction measurement by means of a phoropter, can be determined. Aberration values can be determined from the wavefront measurement, wherein aberration values are one time ascertained for a first extension of the wavefront measurement, in particular for an entire range or diameter of the wavefront measurement, and second aberration values for a subset, which has a second, smaller extension compared to the first extension. Therein, aberration values can be aberrations of the eye, in particular low order aberrations or those up to the second order, which can for example be described by means of Zernike polynomials. These aberration values can then respectively be converted to refractive power errors or diopter values. Thus, one obtains the refractive power portion, which the respective aberration values cause, wherein they are preferably different due to the different extensions of the considered wavefront measurements.

The first and the second extension can be diameters of circular wavefront measurements and/or for example axis distances of ellipses. This means that the extensions can be elliptical and/or circular. The first and the second extension can have an identical centering or a different centering, wherein the centers of the extension can preferably be applied for different reference centers. Thus, a pupil center, a corneal vertex, a corneal apex, a corneal center or an achromatic axis can for example be provided as the reference center.

Subsequently, a difference can be ascertained from the ascertained first refractive power error and second refractive power error, wherein this difference of the refractive effect describes higher order aberrations.

This difference of refractive power errors can then be subtracted from the predetermined subjective refractive power correction. In particular, the difference of the refractive power errors represents higher order aberrations, which cannot be ascertained from the glasses correction measurement, wherein they can be compensated for by additional subtraction from the subjective refractive power correction. Finally, the aberration-corrected refractive power change can then be provided in the form of control data to control the ophthalmological laser for correcting a cornea by means of optical breakthrough. Preferably, artificially induced aberrations can also be introduced in the respective aberration values, for example to treat presbyopia.

The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

By the invention, the advantage arises that improved control data for correcting a visual disorder can be provided, in which optical aberrations can be reduced or avoided.

The invention also comprises embodiments, by which additional advantages arise.

In an embodiment the first aberration values are determined from a wavefront measurement from an entire pupil diameter, in particular a maximum pupil diameter. In other words, the first extension corresponds to the pupil diameter. Herein, it is preferably provided that the pupil diameter is maximized, for example pharmacologically or by adaptation of ambient light conditions, wherein the first aberration values are thus ascertained from the entire pupil and the second aberration values from a partial range thereof.

In a further embodiment a centering of the first extension and of the second extension is different. This means that a central point or centroid of the respective extensions, in particular of the circular extensions, does not coincide, such that the first extension has another central point than the second extension. For example, different reference centers of the eye can be taken into account, which results in an improvement in the determination of aberrations, in particular higher order aberrations.

In a further embodiment the second extension is centered on a predetermined visual axis of the eye. The visual axis can for example be determined by means of preceding measurements and/or the visual axis can be centered based on the corneal vertex, which usually can be used as a reference for the visual axis. Hereby, the advantage arises that a difference of reference centers, in particular a pupil center and the visual axis, can in particular be taken into account by the aberration values, which results in an improvement of the determination of higher order aberrations.

In a further embodiment only low order aberrations are determined for the first and second aberration values. In other words, myopia, hyperopia and astigmatism can be determined as the aberration values. Thus, a comparability of the refractive power errors from the wavefront measurement to those of the subjective refraction exists, since only low order aberrations can also be ascertained in the glasses correction measurement.

In a further embodiment higher order aberrations from the subjective refractive power correction are compensated for by the aberration-corrected refractive power change. In other words, by the effect of rescaling of the wavefront measurement and forming the difference thereof, one achieves the effects of higher order aberrations, which can be subsequently decoupled or compensated for from the subjective refractive power correction.

In a further embodiment the first and second aberration values are ascertained from the wavefront measurements by means of Zernike polynomials, in particular by means of low order Zernike polynomials. Thus, as the representation of the aberrations, which are ascertained from the wavefront measurements, Zernike polynomials can be formed, the orders of which represent the different optical aberrations. Thus, aberrations up to the second order can in particular be ascertained, wherein a difference of the refractive power errors determined therefrom represents an effect of higher order aberrations. Alternatively to Zernike polynomials, further wavefront descriptions, like Fourier polynomials, G-polynomials or wavelets, can also be used for describing the aberration values.

A further aspect of the invention relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate a predefined corneal volume with predefined interfaces of a human or animal eye by means of optical breakthrough, in particular at least partially separate it by means of photodisruption and/or to ablate corneal layers by means of (photo)ablation and/or to effect a laser induced refractive index change in the cornea and/or the eye lens and/or to increase a crosslinking of the cornea.

In a further advantageous configuration of the treatment apparatus according to the invention, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is allowed in the generation of the interfaces. In particular, the range between 700 nm and 780 nm can also be selected as the wavelength range.

In further advantageous configurations of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by means of a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims.

FIG. 1 depicts a schematic representation of a treatment apparatus according to an exemplary embodiment.

FIG. 2 depicts a schematic, quasi perspective view of a cornea.

FIG. 3 depicts a schematic method diagram for providing control data according to an exemplary embodiment.

In the figures, identical or functionally identical elements are provided with the same reference characters.

### DETAILED DESCRIPTION

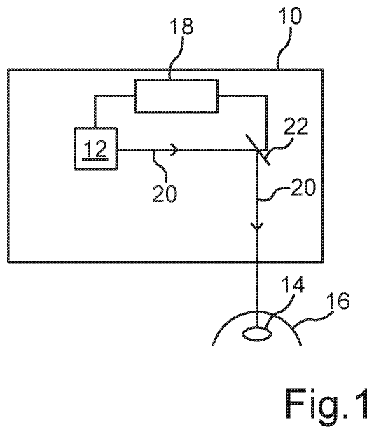

FIG. 1 shows a schematic representation of a treatment apparatus 10 with an eye surgical laser 12 for removing a tissue 14 from a human or animal cornea 16 by means of photodisruption and/or photoablation. For example, the tissue 14 can represent a lenticule or also volume body, which can be separated from the cornea 16 by the eye surgical laser 12 for correcting a visual disorder. A geometry of the tissue 14 to be removed can be provided by a control device 18, in particular in the form of control data, such that the laser 12 emits pulsed laser pulses in a pattern predefined by the control data into the cornea of the eye 16 to remove the tissue 14. Alternatively, the control device 18 can be a control device 18 external with respect to the treatment apparatus 10.

Furthermore, FIG. 1 shows that the laser beam 20 generated by the laser 12 can be deflected towards the cornea 16 by means of a beam deflection device 22, namely a beam deflection apparatus such as for example a rotation scanner, to remove the tissue 14. The beam deflection device 22 can also be controlled by the control device 18 to remove the tissue 14.

Preferably, the illustrated laser 12 can be a photodisruptive and/or photoablative laser, which is formed to emit laser pulses in a wavelength range between 300 nanometers and 1400 nanometers, preferably between 700 nanometers and 1200 nanometers, at a respective pulse duration between 1 femtosecond and 1 nanosecond, preferably between 10 femtoseconds and 10 picoseconds, and a repetition frequency of greater than 10 kilohertz, preferably between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

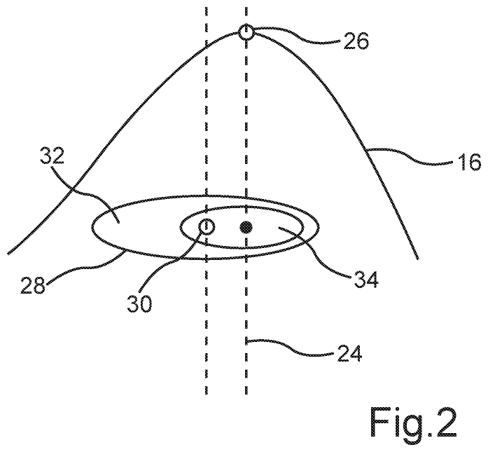

For example, the treatment apparatus 10 can be configured to effect a refractive power change of the cornea 16 by removing the tissue 14, to correct a visual disorder. For the determination of the required refractive power change, different methods can be used, wherein a preferred method is a glasses correction measurement by means of a phoropter, from which a subjective refractive power correction can be determined. This determination is advantageous since the correction can hereby be planned based on the visual axis of the eye. However, it is disadvantageous therein that optical aberrations, in particular higher order aberrations, cannot be ascertained from the glasses correction measurement alone. Hereto, further measurements, in particular wavefront measurements, are provided, wherein the correction of these optical aberrations is explained below with the aid of FIG. 2.

In FIG. 2, a schematic, quasi perspective view of a cornea 16 with a pupil 28 is illustrated. Therein, the eye can comprise multiple optical systems, the centers of which are offset to each other, wherein they overall compose to a visual axis 24, which defines the visual sense of the patient. In particular, the visual axis 24 can be that axis, which defines the visual sense of the patient in the glasses correction measurement and based on which the subjective refractive power correction can be ascertained. Herein, the visual axis can for example be oriented based on a corneal vertex 26, wherein the visual axis 24 can also deviate from the corneal vertex 26. If the refractive power change is planned only due to this visual axis 24, however, it can occur that higher order optical aberrations are not taken into account for the treatment, which for example arise due to a shift of the optical systems of the eye. Thus, the visual axis 24 or the corneal vertex 26 can for example be shifted to a pupil center of the pupil 28, whereby the optical aberrations can arise.

For determining the optical aberrations or aberrations, wavefront measurements are usually provided, which are measured through the pupil 28 and thus have the pupil center 30 as a reference center. However, since the pupil center 30 is herein usually not situated on the visual axis 24, the aberrations from the wavefront measurements cannot be directly related to the subjective refraction.

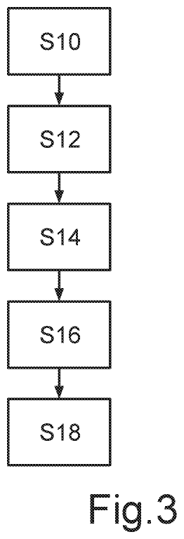

In order to nevertheless consider the optical aberrations, the method shown in FIG. 3 can be performed.

FIG. 3 shows a schematic method diagram for providing control data for an ophthalmological laser 12 of a treatment apparatus 10 for avoiding optical aberrations, in particular higher order aberrations. Therein, the following method steps are explained together with the representation of the cornea 16 shown in FIG. 2.

In a step S10, first aberration values are ascertained from a predetermined wavefront measurement of the eye, wherein the wavefront measurement is performed based on a first extension 32 of the pupil 28. Herein, the first extension 32 can preferably be determined through the entire pupil diameter with a maximized pupil 28. Therein, the first aberration values with this first extension 32 preferably include only low order aberrations like myopia, hyperopia and astigmatism, which can for example also be ascertained from the subjective refractive power correction of the glasses correction measurement. This is advantageous since they can thus be related in a later step. The aberration values can for example be provided in the form of Zernike polynomials. From these first aberration values, a first refractive power error can then be determined, which means that the aberration values can be converted into a refractive power value. For example, the first aberration values can yield that the first refractive power error is 7.25 diopters.

In a step S12, second aberration values can be ascertained from a subset of the entire wavefront measurement, wherein the subset has a second extension 34 or a second diameter. Therein, the second extension 34 is smaller than the first extension 32, which means that only a partial range within the entire wavefront measurement is selected. Therein, the shape of the respective extensions is preferably circular or elliptical. Particularly preferably, it can further be provided that a centering of the first extension 32 and of the second extension 34 differ from each other. Thus, the first extension 32 can for example have the pupil center 30 as a centering or reference center, and the second extension 34 can preferably have the visual axis 24 or an axis of the corneal vertex 26 as the reference center. Therein, the thus ascertained second aberration values preferably also include only low order aberrations, in particular up to the second order.

From these second aberration values, a second refractive power error can then be determined. Herein, the aberrations can for example also be represented by means of Zernike polynomials from the wavefront measurements. However, other wavefront descriptions, such as for example Fourier polynomials, G-polynomials or wavelets, are alternatively also possible.

Due to the different extension, which has been used for determining the aberration values in the wavefront measurements, the aberration values and thus the refractive power errors can differ from each other, wherein this difference arises due to an influence of higher order aberrations, which in particular have not been directly determined. In this example, the second refractive power error can for example be 6.25 diopters.

In a step S14, the difference between the first and the second refractive power error can then be ascertained. Thus, in this example 7.25 diopters−6.25 diopters=1 diopter.

In a step S16, an aberration-corrected refractive power change can be ascertained from the difference of refractive power errors ascertained in step S14 in that this difference is subtracted from a predetermined subjective refractive power correction, which originates from the glasses correction measurement. In this example, the subjective refractive power correction, which can in particular be determined by a phoropter, can for example have a value of 5 diopters. Thus, the aberration-corrected refractive power change is in this example:

$$5 diopters - 1 diopter = 4 diopters .$$

This means that a correction of 4 diopters provides a refractive power change in this example, in which optical aberrations, in particular higher order aberrations, are also taken into account.

Finally, control data for the ophthalmological laser 12 can be provided in a step S18, which includes the aberration-corrected refractive power change, for example the previously mentioned 4 diopters, to separate the tissue 14 from the cornea 16. Thereby, a refraction correction of the cornea 16 is provided, wherein a generation of optical aberrations can be reduced or avoided at the same time.

Overall, the examples show, how an effect of higher order aberrations on the subjective refraction can be compensated for by the invention.

1. A method for providing control data for an ophthalmological laser of a treatment apparatus for avoiding optical aberrations, wherein the method comprises the following steps performed by a control device:
ascertaining first aberration values from a predetermined wavefront measurement of an eye, which has a first extension, wherein a first refractive power error is determined from the first aberration values;
ascertaining second aberration values from a subset of the predetermined wavefront measurement, which has a second extension, wherein the second extension is smaller than the first extension, wherein a second refractive power error is determined from the second aberration values;
ascertaining a difference between the first and the second refractive power error;
ascertaining an aberration-corrected refractive power change by subtracting the ascertained difference of refractive power errors from a predetermined subjective refractive power correction, which is predetermined from a glasses correction measurement; and
providing the control data for the ophthalmological laser, which includes the aberration-corrected refractive power change.

2. The method according to claim 1, wherein the first aberration values are determined from a wavefront measurement from an entire pupil diameter, in particular a maximum pupil diameter.

3. The method according to claim 1, wherein a centering of the first extension and the second extension is different.

4. The method according to claim 1, wherein the second extension is centered on a predetermined visual axis of the eye.

5. The method according to claim 1, wherein only low order aberrations are determined for the first and second aberration values.

6. The method according to claim 1, wherein higher order aberrations from the subjective refractive power correction are compensated for by the aberration-corrected refractive power change.

7. The method according to claim 1, wherein the first and second aberration values are ascertained from the wavefront measurements by means of Zernike polynomials, in particular by means of low order Zernike polynomials.

8. A method for controlling a treatment apparatus, wherein the method includes the following steps:
the method steps of a method according to claim 1, and
transferring the provided control data to a respective ophthalmological laser of the treatment apparatus.

9. A control device, which is configured to perform a respective method according to claim 1.

10. A treatment apparatus with at least one ophthalmological laser for separation of a corneal volume with predefined interfaces of a human or animal eye by means of optical breakthrough, in particular by means of photodisruption and/or photoablation, and at least one control device according to claim 9.

11. (canceled)

12. A non-transitory computer-readable medium, on which a computer program is stored, the computer program including commands, which cause a treatment apparatus to execute a method according to claim 1.
